(19) United States
(12) Patent Application Publication
(10) Pub. No.: US 2014/0248233 A1
(43) Pub. Date: Sep. 4, 2014

(54) BIOCOMPATIBLE COMPOSITE MATERIALS

(71) Applicants: Sandra Downes, York (GB); Anita Kaur Bassi, Derby (GB); Mohsen Zakikhani, Birmingham (GB)

(72) Inventors: Sandra Downes, York (GB); Anita Kaur Bassi, Derby (GB); Mohsen Zakikhani, Birmingham (GB)

(73) Assignee: The University of Manchester, Manchester (GB)

(21) Appl. No.: 13/984,708

(22) PCT Filed: Feb. 8, 2012
(86) PCT No.: PCT/GB2012/000126

§ 371 Date: Jan. 21, 2014

(30) Foreign Application Priority Data
Feb. 11, 2011 (GB) 1102468.4

(51) Int. Cl. A61K 31/765

(52) U.S. Cl. CPC A61K 31/765
USPC 424/78.37

(57) ABSTRACT

An injectable composite material comprising a fibrous material comprised of a fir biocompatible low melting point polyester dispersed in a host medium comprised of second biocompatible low melting point polyester.

The present invention relates to biocompatible composite materials for use in, for example, spinal stabilization and/or fixation procedures.

Biocompatible materials produced by synthetic means are becoming ever more important as new products and methods are sort to treat medical conditions resulting from experiences ranging from acute trauma (e.g. fractures of the vertebrae due to accident) to chronic illness (e.g. osteoporosis, osteogenesis imperfecta or degenerative vertebral disorders such as spondylolisthesis).

In healthy individuals relatively minor fractures to bone resulting from acute trauma usually heal naturally over a short period of time. In more severe cases, a bone graft or implant of some kind may be required. There can be long term clinical problems if the bone defects are exceptionally large. In osteoporotic patients, which is a specific bone condition, the risk of initial fracture is greatly increased as compared to a healthy individual and the ability for the individual to recover naturally is significantly reduced. There is a clinical need for products which could be used in treating such patients.

Osteoporosis is a disease of the bone in which bone mineral density is lower than normal and the microarchitecture and protein structure of the bone is disrupted as a result of homeostatic imbalance between bone formation by cells known as osteoblasts and bone resorption by cells known as osteoclasts. Paget's disease (osteitis deformans) is a chronic illness which is similar to osteoporosis in so far as it also results from an imbalance between bone formation and bone resorption. As a result, there are similarities between the treatments administered to patients suffering from the two conditions.

Currently, there is no commercially available implant designed for patients suffering from osteoporosis and/or Paget's disease that can be implanted at a specific site, for example within the spine, where it is needed. Bone graft substitutes for both normal and osteoporotic patients are currently human/animal derived, or are synthetic scaffolds, however they lack bioactivity and osteoinductivity. Moreover, in conditions requiring spinal fixation, cages and the like are often employed whose outer surface defines a screw thread or set of angled projections to facilitate insertion at the required site and whose interior defines a space for receipt of bone morphogenetic protein (BMP) with the aim of encouraging bone growth into the implant to increase the strength of the connection between the adjacent vertebrae. There are however problems associated with BMPs. For example, in the USA there is limited FDA approval for different types of BMP and in different types of spinal fusion procedure. BMP is currently much more expensive than other bone graft substitutes, and there have also been concerns raised over the use of BMPs in anterior cervical fusion procedures since it has been associated with soft tissue swelling and subsequent restriction of the patient's airway.

An object of the present invention is to obviate or mitigate one or more of the aforementioned problems.

According to a first aspect of the present invention there is provided an injectable composite material comprising a fibrous material comprised of a first biocompatible low melting point polyester dispersed in a host medium comprised of a second biocompatible low melting point polyester.

The present invention provides an improved biocompatible composite material incorporating a fibrous polyester dispersed in a polyester host medium. By using low melting point polyesters in both cases, i.e. polyesters with melting points below around 100° C., the composite material is eminently suitable for use in biomedical applications. The composite material therefore represents a significant improvement over comparable biomaterials, discussed in more detail below, which typically must be delivered to the intended site at temperatures over 100° C., and which therefore present a number of difficulties to the surgical team.

In the Examples presented below a specific low melting point biocompatible polyester, polycaprolactone (PCL), was investigated for use as both the first polyester in the fibrous material of the composite and as the second polyester in the host medium. Tests were conducted using a range of different molecular weights of PCL. It will be appreciated however that the first and second polyesters employed in the composite material according to the present invention do not have to comprise or consist of the same type of polyester or possess the same molecular weight.

In the Examples, injectable PCL-based composite materials were prepared by taking PCL of varying molecular weights and heating it to above its melting point of around 60° C. Electrospun PCL fibrous scaffolds, with and without further functionalisation with a phosphonic acid polymer, were then dispersed into the PCL melt. The different samples were injected into a sawbone, which mimics bone structure, and pull-out tests performed to evaluate the interfacial tensile strength of the PCL-based composite materials according to the present invention and the sawbone. As shown below, the results for the materials according to the present invention were compared with the interfacial tensile strength between the sawbone and a commercially available bone cement. The composite materials according to the present invention exhibited a surprising and unexpectedly large increase in interfacial tensile strength over the commercial material.

In the composite material according to the first aspect of the present invention it is preferred that at least one, more preferably both, of the first and second polyesters is an aliphatic polyester. At least one, preferably both, of the first and second polyesters may exhibit a melting point of less than around 80° C., more preferably a melting point of around 20 to 70° C., and still more preferably a melting point of around 60° C.

In a particularly preferred embodiment of the first aspect of the present invention at least one, preferably both, of the first and second polyesters is polycaprolactone.

The fibrous material may comprise around 1 to 20% (w/w) of at least one, preferably both, of the first and second polyesters, and more preferably may comprise around 10% (w/w) of at least one of the first and second polyesters.

At least one of the first and second polyesters may possess a number average molecular weight of around 20,000 to 150,000 g/mol, more preferably around 30,000 to 110,000 g/mol, and still more preferably a number average molecular weight of around 45,000 g/mol.

The fibre content of the composite material may be around 1 to 30% (w/v), more preferably around 5 to 20% (w/v). The Examples below suggest that a fibre content towards the upper end of the recited ranges may bestow the composite material with a higher interfacial strength. In applications where a high interfacial strength is a particularly important property of the composite material it is therefore preferred that the fibre content of the composite material is towards the top end of the recited ranges, for example, around 10 to 25% (w/v), or around 15 to 22% (w/v).

The number average molecular weight of the first polyester may be approximately the same as the number average molecular weight of the second polyester. Alternatively, the number average molecular weight of the first polyester may be different to the number average molecular weight of the second polyester depending upon the desired properties of the final composite material incorporating the first and second polyesters.

While fibres of any size and/or shape may be utilised in the fibrous material within the composite material of the present invention it is preferred that the fibres within the fibrous material possess a sub-micron average diameter, for example, an average diameter in the range of around 10 to 1000 nm, more preferably around 100 to 500 nm, and yet more preferably around 200 to 400 nm. It is particularly preferred that the diameters possess an average diameter of around 250 to 300 nm.

The fibres within the fibrous material may have a substantially ordered arrangement, partly ordered arrangement or be essentially randomly arranged. It is particularly preferred that the fibres are generally aligned since this increases the strength of the final composite material. In the Examples below, images at different levels of magnification are provided which show how well aligned the fibres of the fibrous material are within the host medium.

The fibrous material is preferably porous. This may be advantageous since it should allow infiltration of cells throughout the structure of the fibrous material and affords a significantly increased specific surface area for cell growth as compared to non-porous materials. Porous fibrous materials also facilitate the penetration of nutrients to growing cells supported by the fibrous materials. Additionally, providing the fibrous material with a porous structure affords a means to manipulate and control various physical, chemical and biological properties of the fibrous material so that they can be tailored to suit a particular application.

It is preferred that at least one, and possibly two, three, four or more, type of phosphonic acid polymer is comprised in the fibrous material, the host medium or both the fibrous material and the host medium.

The or each phosphonic acid polymer may be incorporated into the material of the fibres of the fibrous material and/or provided as a coating over at least a region of the fibrous material. Thus, some or all of the fibres of the fibrous material may be formed from one or more types of phosphonic acid polymer and/or some or all of the fibres of the fibrous material may be provided with a coating containing one or more types of phosphonic acid polymer. Moreover, the fibres may be formed from or contain one type of phosphonic acid polymer and the coating may be formed from or contain a different type of phosphonic acid polymer. In addition, phosphonic acid moieties may be formed by means of chemical reaction on the surface of the fibrous material or by functionalisation of the fibrous material with an appropriate molecule containing phosphonic acid moieties.

The or each phosphonic acid polymer may be incorporated into the host medium in any convenient manner, for example by co-polymerisation with the second polyester, and optionally with any other polymers that are intended to be present in the host medium, or, by way of further example, by mixing, blending or combining the phosphonic acid polymer(s) with the second polyester, and optionally with any other polymers that are intended to be present in the host medium.

Reference herein to a “phosphonic acid polymer” encompasses any type of polymer produced by polymerisation of a monomer incorporating a phosphonic acid group and a polymerisable moiety (e.g. a vinyl group), optionally in combination with one or more other types of monomer. It will also be appreciated that reference herein to a “phosphonic acid polymer” also encompasses polymers of phosphonic acid salts. Moreover, the aforementioned polymers may comprise at least one phosphono or phosphino moiety.

In preferred embodiments in which the fibrous material and/or host medium comprises one or more types of phosphonic acid polymer, the fibrous material and/or host medium incorporates phosphorus atoms as pendant groups along a polymer backbone. This is analogous to the active moiety in bisphosphonate drugs used to treat conditions such as osteoporosis and Paget's disease. The composite material of the present invention thus possesses the ability to bind to the predominant constituent of bone, hydroxyapatite, and to be internalised by resorbing osteoclasts. Chemical analysis of a tissue scaffold comprising a related material, which is the subject of the applicant's co-pending International patent application no. PCT/GB2010/001482, indicated that calcium was concentrated in areas where there was an increased phosphorous concentration suggesting that there was binding between phosphorous and calcium. In vitro and in vivo studies demonstrated cellular interactions with the scaffold leading to cell attachment and proliferation and the formation of extracellular matrix.

In the composite material of the present invention the fibrous material and/or the host medium may comprise one or more types of phosphonic acid polymer in any suitable amount. It is preferred that the total amount of said at least one type of phosphonic acid polymer comprised in the composite material is around 0.1 to 30% (w/v), more preferably around 1 to 20% (w/v). The total amount of phosphonic acid polymer may be derived from just the fibrous material where only the fibrous material comprises any phosphonic acid polymer. Alternatively, all of the phosphonic acid present in the composite material may be provided in the host medium. Or, as a further alternative, both the fibrous material and the host medium may contain some phosphonic acid polymer, which may be the same type(s) of phosphoric acid polymer in substantially similar amounts and having substantially similar molecular weights, or the type, amount and/or molecular weight of the phosphonic acid polymer(s) in the fibrous material may be different to those in the host medium.

In a preferred embodiment a first phosphoric acid polymer is comprised in the fibrous material. The first phosphoric acid polymer may be chemically attached to fibres within the fibrous material and/or may be comprised in a mixture, blend or combination of the first polyester and the first phosphoric acid polymer. The fibrous material may comprise around 0.1 to 30% (w/v) of the first phosphoric acid polymer, more preferably around 1 to 20% (w/v) of the first phosphonic acid polymer.

In another preferred embodiment a second phosphonic acid polymer is comprised in the host medium. The host medium may comprise around 0.1 to 30% (w/v) of the second phosphonic acid polymer, or more preferably around 1 to 20% (w/v) of the second phosphoric acid polymer.

The starting material that is formed into phosphoric acid polymer(s) incorporated into the composite material of the invention may be a phosphonic acid oligomer, homopolymer or heteropolymer (e.g. copolymer, terpolymer, etc), or may be a mixture or blend of any such phosphonic acid moieties in combination with one or more other polymers. The molecular weight of the polymer could be that of the combination of a few monomer units forming the polymer (as defined by the regulatory bodies as “polymer”) to several million depending upon the intended application of the polymer. More preferably the molecular weight is in the range of around 100 g/mol to 500,000 g/mol, and most preferably the molecular weight is in the range of around 100 g/mol to 200,000 g/mol.

In a preferred embodiment the phosphoric acid polymer is derived from a homopolymer of a phosphoric acid monomer, such as vinyl phosphoric acid (VPA). Alternatively, the phosphonic acid polymer may be formed from a co-polymer of a phosphonic acid monomer copolymerised with a second type of polymerisable monomer. Preferred copolymerisable monomers include polymerisable carboxylic acid monomers, such as acrylic acid and methacrylic acid (referred to herein generically as “(meth)acrylic acid”) monomers.

In accordance with preferred embodiments of the present invention, the polymer may incorporate an active moiety selected from a phosphono-component and/or phosphino-component. The phosphono-component or phosphino-component may consist essentially of vinylphosphonic acid (VPA), vinylidene-1,1-diphosphonic acid (VDPA), a phosphono substituted mono- or di-carboxylic acid, hypophosphorus acid or a salt, such as an alkali metal salt of hypophosphorus acid. By way of example, the phosphono-component may consist essentially of a homopolymer of VPA, VDPA, or phosphono-succinic acid.

The composition of the composite material may further incorporate one or more additional components, such as unsaturated sulphonic acid, saturated or unsaturated carboxylic acids, unsaturated amides, primary or secondary amines, polyalkylene imines, or amine-terminated polyalkylene glycols. For example, the polymeric composition may consist essentially of a copolymer of vinylphosphonic acid (VPA) with vinylsulphonic acid (VSA), or with acrylic acid (M), methacrylic acid (MAA) or acrylamide. Alternatively, the polymeric composition may consist essentially of a copolymer of VDPA with VSA, or with AA, MAA or acrylamide. As another example, the polymeric composition may consist essentially of a terpolymer of VDPA, VSA and either AA, MMA or acrylamide. Alternatively, the polymeric composition may consist essentially of the reaction product VPA, or VDPA, or a mixture of VPA and VDPA, and any one of the following:

a. a primary amine;
b. a secondary amine;
c. a polyethylene imine;
d. an amine terminated polyethylene or polypropylene glycol (e.g. jeffamines); and/or
e. a hypophosphorus acid or salt thereof.

A particularly preferred copolymer for use in fabricating fibres for the fibrous material and/or incorporation into the host medium is poly (vinyl phosphonic acid-co-acrylic acid) (P(VPA-Co-M)).

It will be appreciated that any of the aforementioned polymers, copolymers or terpolymers may comprise at least one phosphono or phosphino moiety.

The fibrous material and/or host medium in the composite material according to the present invention may incorporate one or more biocompatible polymers in addition to the first and second low melting point polyesters. Preferably fibres of the fibrous material and/or the host medium are made from or comprise polycaprolactone (poly-ε-caprolactone; or PCL). PCL is a biocompatible material that is approved by the US Food and Drug Administration (FDA) for use in biomedical applications and is therefore eminently suitable for application in composite materials according to the present invention. In accordance with the present invention, a suitable amount of one or more other biocompatible polymers may be incorporated into the composite material of the invention such as poly(glycolic acid), poly(lactic acid), poly(lactic-co-glycolic acid), and/or poly(methyl methacrylate).

In a particularly preferred embodiment of the first aspect of the present invention there is provided an injectable biocompatible composite material comprising:
a host medium comprising PCL and P(VPA-Co-AA); and
a fibrous material formed from PCL and P(VPA-Co-AA), or PCL with P(VPA-Co-AA) chemically attached thereto, dispersed in said host medium.

In a further preferred embodiment of the present invention the composite material further comprises an ionic compound, for example, a radiopaque compound of the kind used in radioimaging, such as barium sulphate and/or zirconium oxide. The composite material may comprise any desirable amount of a radiopaque compound, such as around 1 to 50 wt %, or more preferably around 10 to 40 wt %. With reference to the Examples below it has unexpectedly been observed that adding even a relatively low amount of a radiopaque compound, such as barium sulphate (5 wt %) to the composite material of the present invention has a significant effect on the interfacial tensile strength of the composite.

A second aspect of the present invention provides a method for preparing an injectable composite material comprising a fibrous material comprised of a first biocompatible low melting point polyester dispersed in a host medium comprised of a second biocompatible low melting point polyester, the method comprising heating the host medium to a temperature above its melting point and dispersing the fibrous material in the molten host medium.

A third aspect of the present invention provides a method for providing a composite material at a target site, the composite material comprising a fibrous material comprised of a first biocompatible low melting point polyester dispersed in a host medium comprised of a second biocompatible low melting point polyester, the method comprising delivery to the target site of the host medium when in a molten state with the fibrous material dispersed within said molten host material and then curing said host medium and/or fibrous material.

Curing may be effected by any appropriate means, but a particularly suitable method is to effect curing by cooling the host medium and/or fibrous material to below its melting point.

Curing is preferably effected as quickly as possible following delivery to the target site. It is preferred that curing is effected in less than 5 minutes, more preferably in around 30 seconds to 3 minutes, and most preferably in around 90 seconds. This lowest curing time has been achieved in an exemplary embodiment of the composite material of the present invention as explained below in the Examples.

It is preferred that delivery of the composite material is effected in such a manner so as to generally align fibres of the fibrous material at the target site. A simple and convenient method of delivery is injection.

Suitable target sites for delivery of the composite material of the present invention include a site defined by a spinal fixation implant, for example, a cage-type spinal fusion device whose interior defines a space for receipt of an amount of the composite material of the present invention. The cage can then be inserted by the surgeon at the appropriate surgical site and the composite material then help to anchor the cage in place and encourage bone growth into the composite. Bone growth is particularly encouraged by embodiments in which the fibrous material and/or host medium of the composite includes one or more types of phosphonic acid polymer. This latter features also contributes to increasing the strength of the connection between the implant and adjacent vertebrae as suggested by the results presented in the Examples below where addition of even a relatively small amount (1.5%) of phosphonic acid polymer to the fibrous material and host medium increased the interfacial tensile strength of the composite over composites not including any phosphonic acid polymer.

The target site may itself be a location in or adjacent to the spine of a human or animal. That is, it is envisaged that the composite material of the present invention can be administered directly to a site defined by the spine of a patient where previously a bone cement or the like had been used to effect fixation or stabilization. A significant advantage of using a composite material according to a preferred embodiment of the present invention in which both the fibrous material and the host medium comprise PCL, is that, by virtue of PCL's low melting point (around 60° C.), the composite material can be melted by heating to only around 80° C. and then injected directly into the target site while still molten. Not only is injection a simple and convenient method of administration, the process of injection serves to align fibres of the fibrous material during delivery to the target site which increases the strength of the final, cured composite material.

A fourth aspect of the present invention provides a method for treating a spinal condition in a patient requiring spinal stabilisation or fixation at a target site in or adjacent to the spine of the patient, wherein the method comprises providing a composite material at said target site, the composite material comprising a fibrous material comprised of a first biocompatible low melting point polyester dispersed in a host medium comprised of a second biocompatible low melting point polyester, the method comprising delivery to the target site of the host medium when in a molten state with the fibrous material dispersed within said molten host material and then curing said host medium and/or fibrous material.

A fifth aspect of the present invention provides a medical device for spinal stabilization or fixation comprising an implant and an injectable composite material according to the first aspect of the present invention. The implant may define a space for receipt of the injectable composite material.

A sixth aspect of the present invention provides an injectable medicament for use in the treatment of a spinal condition in a patient requiring spinal stabilisation or fixation, said medicament comprising an injectable composite material according to the first aspect of the present invention.

Preferred exemplary embodiments of the present invention will now be described by way of example only, in which:

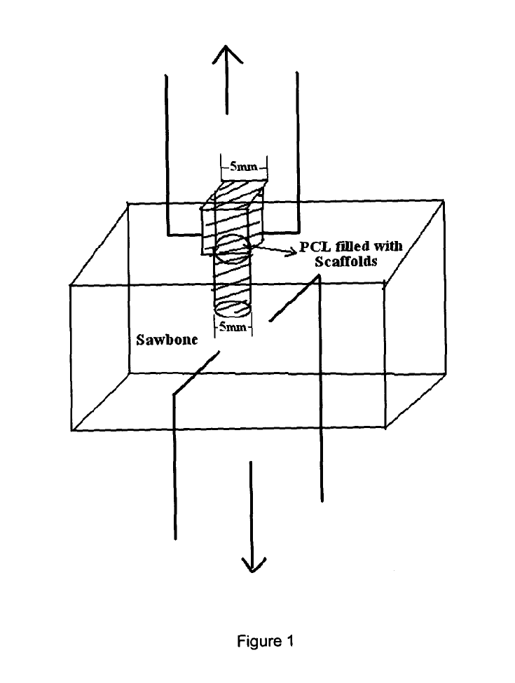

FIG. 1 is a schematic diagram of the apparatus used for pull-out experiments to determine the interfacial tensile strength of various materials including composite materials according to the present invention and sawbone;

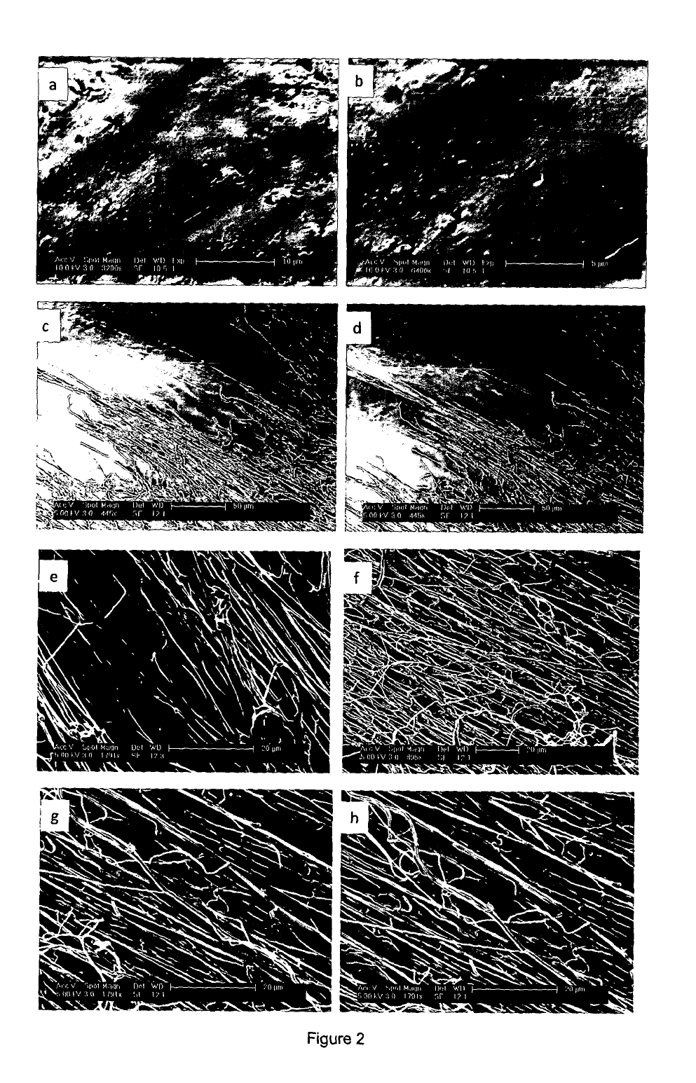

FIGS. 2 (a) and (b) are scanning electron microscope (SEM) images of a surface of a cured PCL-containing polymer not including any fibrous material and therefore not in accordance with the present invention.

FIG. 2 (c) to (h) are SEM images of a cured PCL-containing polymer containing a PCL-based fibrous material, i.e. a cured composite material according to the present invention;

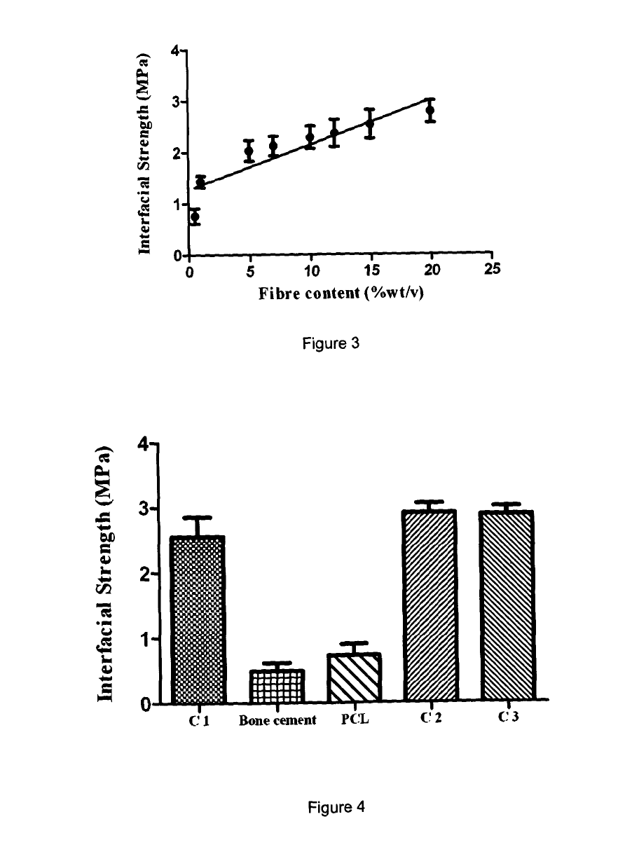

FIG. 3 is a graph of how fibre content related to interfacial tensile strength. Linear regression fitting was used to fit the values. Data are reported as mean±standard deviation (SD). n=6, p<0.0001 means the slope is significantly non-zero;

FIG. 4 is a graph of interfacial tensile strength measured for a range of different materials including a commercial bone cement, a PCL polymer control (i.e. PCL polymer containing no fibrous material), and composite materials according to the present invention. The interfacial strength are significant different between these three materials and bone cement. n=6 p<0.000;

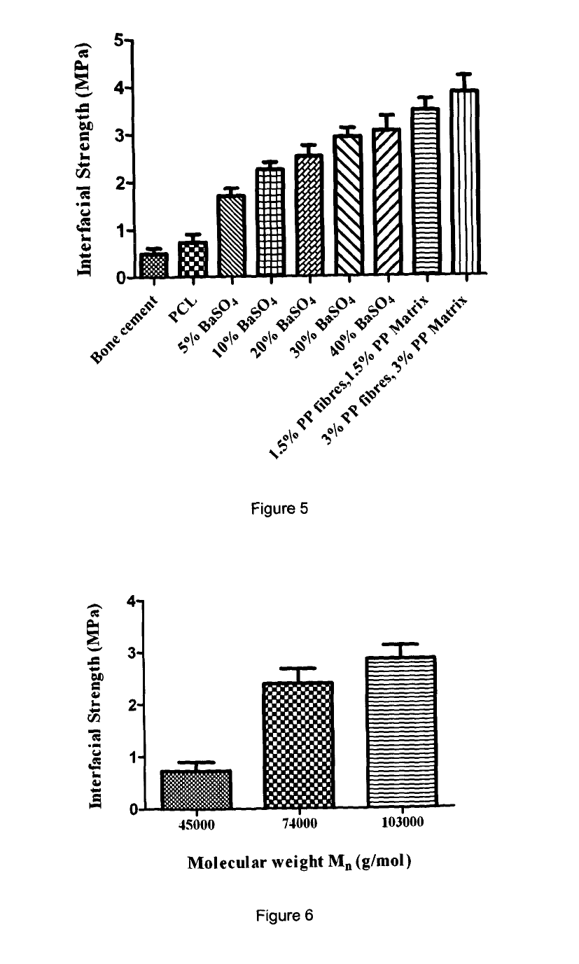

FIG. 5 is a graph of interfacial tensile strength measured for a range of different materials including a commercial bone cement, a PCL polymer control (i.e. PCL polymer containing no fibrous material), and composite materials according to the present invention, a subset of which included different amounts of barium sulphate. The interfacial strength are significant different between these three materials and bone cement. n=6 p<0.000;

FIG. 6 is a graph of interfacial tensile strength measured for different molecular weight PCL-based polymers and sawbone. n=6 p<0.0001;

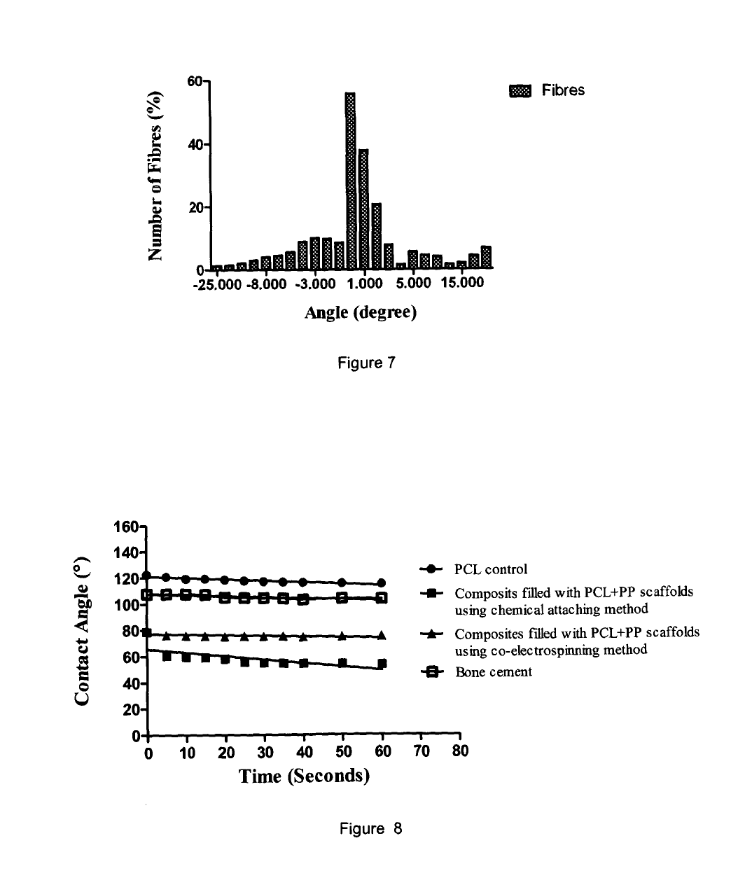

FIG. 7 is a graph showing the distribution in alignment of the fibres in a PCL-based composite material according to the present invention, alignment being caused primarily by injection of the composite material from a syringe;

FIG. 8 is a graph of contact angle for various materials, including a commercial bone cement, a PCL polymer control (i.e. PCL polymer containing no fibrous material), and composite materials according to the present invention. Data are reported as mean±standard deviation (SD) n=6;

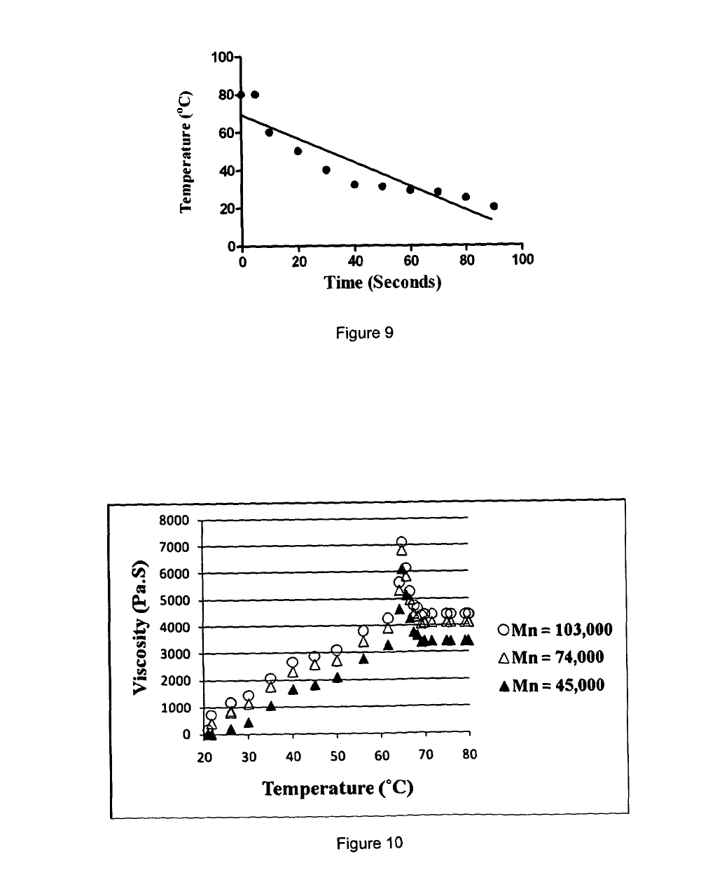

FIG. 9 is a graph of temperature changes with time for a PCL-based composite material according to the present invention. Linear regression fitting was used to fit the points; and

FIG. 10 is a graph of viscosity against temperature for PCL-based polymers possessing three different number average molecular weights (Mₙ). n=6 p<0.0001.

### EXAMPLES

### Step 1: Electrospinning of PCL Fibrous Materials

Polycaprolactone (PCL) (Mₙ=103,000 g·mol⁻¹) determined by gel permeation chromatography (GPC) (Applied Chromatography Systems Ltd) was selected and purchased from Sigma-Aldrich, UK. A 10% (w/v) polymer solution was prepared by dissolving PCL in acetone at 50° C. and continuously stirred for 12 hours.

A concentration of 10% (wt/wt) PCL solution was placed into a 10 ml plastic syringe connected with a stainless steel needle. A voltage of 20 kV was applied between the needle tip and a ground electrode. A 50 μl/min flow rate was used and the distance between the needle tip and the ground electrode was kept at 15 cm. The electrospun PCL fibres were deposited on to an aluminium foil which was fixed on to the ground electrode. After 20 minutes a fibrous material with an average thickness of 66 μm was formed. The fibrous material was then removed from the aluminium foil with care.

The electrospun PCL fibrous materials were also functionalised using a phosphonate polymer (poly (vinyl phosphonic acid-co-acrylic acid); P(VPA-Co-AA)) by two different methods: chemical attachment using both hydrolysis and esterification reactions; and co-electrospinning.

### Step 2: Heating PCL Polymer to a Suitable Temperature

PCL pellets (PCL Mₙ=103,000 g·mol⁻¹ and 45,000 g·mol⁻¹) were put into an oven and heated at a temperature of 80° C. for 12 hours. After heating the PCL polymer melt was transferred into a 2 ml plastic syringe with a 2 mm size nozzle. The PCL polymer was observed to be injectable from the nozzle. After injection, the temperature of the PCL polymer was immediately dropped to 60° C. The polymer was cured after 90 seconds.

### Step 3: Breakdown the PCL Scaffold to Smaller Size

In order to form samples of the injectable composite material for comparative testing, samples of the PCL fibrous material, with and without functionalisation, were cut into 5 mm×5 mm size small pieces.

### Step 4: Inject PCL-Based Composites

The small sized pieces of the PCL fibrous material were added into the PCL polymer melt and stirred using a metal spoon at 80° C. in order to disperse the fibrous material throughout the polymer melt. The composite materials formed were then rolled up to into a column shape and put back into an oven to ensure that the temperature of the composite material was 80° C.

The composites were then transferred into a 2 ml plastic syringe with a 2 mm nozzle. The syringe was immediately taken out of the oven at 80° C. and the composites injected into a sawbone made of cellular rigid polyurethane foam with a 5 mm×5 mm×5 mm size hole and a 5 mm×5 mm size mould above the sawbone surface as shown in FIG. 1. This apparatus was used to carry out the pull-out tests described in more detail below. After injection the temperature of the composites was immediately dropped to 60° C. and the composites were cured after 90 seconds.

### Step 5: Pullout Tests

An Intron 1122 machine was used to apply the necessary force to each sample to measure the interfacial tensile bonding strength between each sample and the sawbone. Each sample was subjected to a pulling force that was perpendicular to the top surface of the sawbone at a crosshead speed of 20 mm/min.

Investigation into Fibre Structure

FIGS. 2 (a) and (b) are SEM images showing a cured PCI polymer surface after injection. This therefore acted as a control. FIGS. 2 (c) and (d) are SEM images showing the cured PCL polymer containing PCL. fibrous material. FIG. 2 (e) is an SEM image showing how the PCL scaffold started to melt into the PCL polymer due to the experimental temperature of 80° C. FIG. 2 (f) to (h) are SEM images showing how the PCL fibres within the fibrous material are aligned due to the mechanical pulling forces applied to the composite during injection of the composite into the pull-out test apparatus.

From FIG. 2 (a) to (h) it can be observed that electrospun PCL fibres were present in the composite material and that they started melting due to the high experimental temperature of 80° C. used during the pull-out tests. The alignment of the fibres was due to the shear force to which the fibres are exposed during injection into the test apparatus. This is promising as the orientation of the PCL fibres will enhance the mechanical properties of the PCL based composites.

### Pull-Out Tests

The relation between the interfacial bonding strength and the amount of the fibres in the PCL-based composites was studied.

In composites according to the invention it was observed that during the pull-out tests sawbone around the injected composite was fractured and pulled out together with the composites. The interfacial tensile bonding strength between the sawbone and the composites according to the invention was calculated as 2.6±0.4 MPa, which is larger than the force required to fracture the sawbone.

A pure PCL polymer control (PCL Mₙ≈45,000 g·mol⁻¹) was melted at 80° C. and a 2 mm thickness film formed after cooling down. The tensile strength of the PCL film was tested using same Instron machine at a crosshead speed of 50 mm/min. The tensile strength of the PCI film control was calculated as 25.2±3.8 MPa.

FIG. 3 shows that the interfacial tensile bonding strength between the PCL-based composite according to the present invention and the sawbone increased as the fibre content increased from one sample to another.

FIG. 4 shows the results of a series of pull-out tests conducted to test the interfacial tensile strength of a series of different materials and sawbone. “PCL” refers to a PCL polymer control in which the PCL has a Mₙ=45,000. “C1” refers to a PCL-based composite material according to the present invention but containing no phosphonic acid polymer. “C2” refers to a PCL-based composite material according to the invention including P(VPA-Co-AA) connected to the PCL fibres using chemical attachment. “C3” refers to a composite material similar to C2 but in which the P(VPA-Co-AA) was co-electrospun with the PCL to form the fibres in the fibrous material. In C1, C2 and C3, PCL polymer with Mₙ≈45,000 g·mol⁻¹ was used as the host medium. In the results shown in FIG. 7, n=6 p<0.0001.

The pull-out test results shown in FIG. 4 show that the strength used to pull the PCL control out of sawbone (“PCL”) was 0.73±0.17 MPa. The strength used to pull the PCL composite including chemically attached phosphoric acid (“C2”) was 2.89±0.12 MPa. The strength used to pull the other PCL composite (“C3”) was 2.68±0.23 MPa. The interfacial bonding strength used to pull the bone cement out of the sawbone was 0.53±0.2 MPa.

It has been reported that bone breakage has been observed at the tensile strength of 1.37-1.47 MPa. The interfacial bonding strength for the polymer composites according to the present invention and the sawbone is therefore much higher, being 2.68±0.23 MPa and 2.89±0.12 MPa. It has been shown that the interfacial tensile strength between a PMMA based bioactive bone cement and bone after 4 weeks implantation is 0.35 MPa. Therefore the PCL-based composite materials according to the present invention exhibited superior results than the PMMA based bioactive bone cement. Statistical evaluation of the data shown in FIG. 4 was performed using GraphPad™ software package. Data are reported as mean±standard deviation (SD) at a significant level of p<0.0001. One-way ANOVA with repeated measures was carried out to compare the groups.

FIG. 5 depicts the results of pull-out tests performed on a further series of different materials and sawbone. In the results shown in FIG. 5 n=6 p<0.0001
“PCL” refers to a PCL polymer control with Mₙ≈45,000 g·mol⁻¹.
“5% BaSO₄” refers to a composite according to the invention comprising 5 wt % barium sulphate added into PCL with Mₙ=45,000 g·mol⁻¹.
“10% BaSO₄” refers to a composite according to the invention comprising 10 wt % barium sulphate added into PCL with Mₙ=45,000 g·mol⁻¹.
“20% BaSO₄” refers to a composite according to the invention comprising 20 wt % barium sulphate added into PCL with Mₙ≈45,000 g·mol⁻¹.
“30% BaSO₄” refers to a composite according to the invention comprising 30 wt % barium sulphate added into PCL with Mₙ=45,000 g·mol⁻¹.
“40% BaSO₄” refers to a composite according to the invention comprising 40 wt % barium sulphate added into PCL with Mₙ≈45,000 g·mol⁻¹.
“1.5% PP fibres, 1.5% PP Matrix” refers to a composite according to the invention including co-electrospun PCL fibres with 1.5% P(VPA-Co-AA) and adding 1.5% P(VPA-Co-AA) in the PCL host medium (PCL Mₙ≈45,000 g·mol⁻¹).
“3% PP fibres, 3% PP Matrix” refers to a composite according to the invention including co-electrospun PCL fibres with 3% P(VPA-Co-AA) and adding 3% P(VPA-Co-AA) in the PCL host medium (PCL Mₙ=45,000 g·mol⁻¹).

As can be seen, adding increasing amounts of barium sulphate increased the interfacial tensile strength of the materials, as did including increasing amounts of a phosphonic acid polymer. It is envisaged that the effect of the phosphonic acid polymer addition would be increased still further in real bone rather than the synthetic sawbone plastic in which these tests were carried out.

Different molecular weight PCL was used to study the interfacial bonding strength between PCL and the sawbone. The results are shown in FIG. 6, in which it can be observed that the interfacial bonding strength increases from 0.73±0.17 MPa to 2.7±0.41 MPa by increasing the PCL molecular weight from Mₙ≈45,000 g·mol⁻¹ to Mₙ≈103,000 g·mol⁻¹. Data are reported as mean±standard deviation (SD) at a significant level of p<0.0001. One-way ANOVA with repeated measures was carried out to compare the groups.

### Fibre Alignment

The alignment of the fibres in the PCL composites resulting from injection of each sample was measured using ImageJ software. FIG. 7 demonstrates how well aligned the fibres of the composite were as a result of the shear force applied to them during the injection process.

### Water Contact Angle

Contact angle measurements were conducted for commercial bone cement, a PCL polymer control and PCL-based composites according to the invention functionalised to include a phosphonic acid polymer by two different methods. The results are shown in FIG. 8.

The contact angle value of water on the surface of the PCL polymer control was 122°, indicating a hydrophobic surface.

The water contact angle on the bone cement was about 110°, which indicated that the surface of the bone cement was also hydrophobic.

In contrast, the contact angle of water on the surface of the each of the two differently functionalised PCL-based composite materials according to the invention was less than 90°, indicating that the composites had surfaces that were hydrophilic. The contact angle value for the surface of each of the two differently functionalised PCL composites was between 60° to 80°, which is much lower than the contact angle of the bone cement. This result suggests that there is good contact and adhesion between the functionalised PCL composites according to the invention and the sawbone.

### Temperature Profile of Injected Composite Material

FIG. 9 shows the temperature change with time when injecting a PCL-based composite material according to the invention into a target site. The total amount of time elapsed from the start of the injection process to when the composite material was cured was 90 seconds.

Viscosity Variation with Temperature

FIG. 10 depicts the results if an investigation into how the viscosity of three different samples of PCL varied with temperature. As can be seen, all three samples followed a very similar profile, with the lowest molecular weight polymer exhibiting a lower viscosity than the other two polymers at similar temperatures.

1. An injectable composite material comprising a fibrous material comprised of a first biocompatible low melting point polyester dispersed in a host medium comprised of a second biocompatible low melting point polyester, wherein at least one type of phosphonic acid polymer is comprised in the fibrous material and/or the host medium.

2-53. (canceled)

54. An injectable composite material according to claim 1, wherein at least one of the first and second polyesters is an aliphatic polyester.

55. An injectable composite material according to claim 1, wherein at least one of the first and second polyesters exhibits a melting point of less than around 80° C.

56. An injectable composite material according to claim 1, wherein at least one of the first and second polyesters is polycaprolactone.

57. An injectable composite material according to claim 1, wherein the fibrous material comprises around 1 to 20% (w/w) of at least one of the first and second polyesters.

58. An injectable composite material according to claim 1, wherein at least one of the first and second polyesters possesses a number average molecular weight of around 20,000 to 150,000 g/mol.

59. An injectable composite material according to claim 1, wherein the fibre content of the composite material is around 1 to 30% (w/v).

60. An injectable composite material according to claim 1, wherein fibres within the fibrous material possess a sub-micron average diameter.

61. An injectable composite material according to claim 1, wherein the fibrous material is porous.

62. An injectable composite material according to claim 1, wherein the total amount of said at least one type of phosphonic acid polymer comprised in the composite material is around 0.1 to 30% (w/v).

63. An injectable composite material according to claim 1, wherein a first phosphonic acid polymer is comprised in the fibrous material.

64. An injectable composite material according to claim 1, wherein a second phosphonic acid polymer is comprised in the host medium.

65. An injectable composite material according to claim 1, wherein the or each phosphonic acid polymer is a phosphonic acid homopolymer, or a phosphonic acid heteropolymer incorporating one or more other types of polymer.

66. An injectable composite material according to claim 1, wherein the or each phosphonic acid polymer is poly(vinyl phosphonic acid-co-acrylic acid).

67. An injectable composite material according to claim 1, wherein the number average molecular weight of the or each phosphonic acid polymer is in the range of around 100 g/mol to 500,000 g/mol.

68. An injectable composite material according to claim 1, wherein the composite material further comprises barium sulphate.

69. An injectable composite material according to claim 68, wherein the composite material comprises around 1 to 50 wt % of a radiopaque compound.

70. A method for preparing an injectable composite material comprising a fibrous material comprised of a first biocompatible low melting point polyester dispersed in a host medium comprised of a second biocompatible low melting point polyester, wherein at least one type of phosphonic acid polymer is comprised in the fibrous material and/or the host medium, the method comprising heating the host medium to a temperature above its melting point and dispersing the fibrous material in the molten host medium.

71. A method for providing a composite material at a target site, the composite material comprising a fibrous material comprised of a first biocompatible low melting point polyester dispersed in a host medium comprised of a second biocompatible low melting point polyester, wherein at least one type of phosphonic acid polymer is comprised in the fibrous material and/or the host medium, the method comprising delivery to the target site of the host medium when in a molten state with the fibrous material dispersed within said molten host material and then curing said host medium and/or fibrous material.

72. A method according to claim 71, wherein curing is effected by cooling the host medium and/or fibrous material to below its melting point.

73. A method according to claim 71, wherein delivery is effected so as to generally align fibres of the fibrous material at the target site.

74. A method according to claim 71, wherein delivery is effected by injection.

75. A method according to claim 71, wherein said target site is defined by a spinal fixation implant.

76. A method according to claim 71, wherein said target site is a location in or adjacent to the spine of a human or animal.

77. A method for treating a spinal condition in a patient requiring spinal stabilisation or fixation at a target site in or adjacent to the spine of the patient, wherein the method comprises providing a composite material at said target site, the composite material comprising a fibrous material comprised of a first biocompatible low melting point polyester dispersed in a host medium comprised of a second biocompatible low melting point polyester, wherein at least one type of phosphonic acid polymer is comprised in the fibrous material and/or the host medium, the method comprising delivery to the target site of the host medium when in a molten state with the fibrous material dispersed within said molten host material and then curing said host medium and/or fibrous material.

78. A medical device for spinal stabilization or fixation comprising an implant and an injectable composite material according to claim 1.

79. An injectable medicament for use in the treatment of a spinal condition in a patient requiring spinal stabilisation or fixation, said medicament comprising an injectable composite material according claim 1.
